(19) United States
(12) Patent Application Publication
(10) Pub. No.: US 2025/0099238 A1
(43) Pub. Date: Mar. 27, 2025

(54) SUTURE PATTERN FOR TENT HEIGHT REDUCTION IN A HEART VALVE PROSTHESIS

(71) Applicant: Medtronic, Inc., Minneapolis, MN (US)

(72) Inventors: Yogesh A. Darekar, Irvine, CA (US); Justin G. Goshgarian, Santa Rosa, CA (US); Brenda Laurie McIntire, Santa Rosa, CA (US); Tiffany T. Dang, Garden Grove, CA (US); Phoebe M. Makar, Fountain Valley, CA (US)

(21) Appl. No.: 18/730,817

(22) PCT Filed: Jan. 20, 2023
(86) PCT No.: PCT/IB2023/050499

§ 371 Date: Jul. 22, 2024

Related U.S. Application Data
(60) Provisional application No. 63/304,224, filed on Jan. 28, 2022.

(51) Int. Cl. A61F 2/24

(52) U.S. Cl. CPC A61F 2/2418; A61F 2/2415; A61F 2220/0075; A61F 2240/001

(57) ABSTRACT

A heart valve prosthesis including a frame and a skirt. The frame including an inner portion configured to support a prosthetic valve component, and an outer portion coupled to the inner portion, the outer portion surrounding the inner portion and configured to anchor the prosthesis. The outer portion including a plurality of first crowns positioned around a first end of the outer portion and a plurality of second crowns positioned inward of the plurality of first crowns. The inner and outer portions are coupled at a plurality of pairs of adjoining crowns. The skirt is disposed within and is coupled to the outer portion, wherein an end segment of the skirt, disposed proximate to the first end of the outer portion, does not substantially extend past an endmost node of the outer portion so as to leave unobstructed a blood flow passageway through each pair of adjoining crowns.

### FIELD

The present technology is generally related to heart valve prostheses implantable via minimally invasive procedures, and in particular is directed to mitral valve prostheses having a low profile.

### BACKGROUND

The human heart is a four chambered, muscular organ that provides blood circulation through the body during a cardiac cycle. The four main chambers include the right atrium and right ventricle which supplies the pulmonary circulation, and the left atrium and left ventricle which supplies oxygenated blood received from the lungs into systemic circulation. To ensure that blood flows in one direction through the heart, atrioventricular valves (tricuspid and mitral valves) are present between the junctions of the atrium and the ventricles, and semi-lunar valves (pulmonary valve and aortic valve) govern the exits of the ventricles leading to the lungs and the rest of the body. These valves contain leaflets or cusps that open and shut-in response to blood pressure changes caused by the contraction and relaxation of the heart chambers. The valve leaflets move apart from each other to open and allow blood to flow downstream of the valve, and coapt to close and prevent backflow or regurgitation in an upstream manner.

Diseases associated with heart valves, such as those caused by damage or a defect, can include stenosis and valvular insufficiency or regurgitation. For example, valvular stenosis causes the valve to become narrowed and hardened which can prevent blood flow to a downstream heart chamber from occurring at the proper flow rate and may cause the heart to work harder to pump the blood through the diseased valve. Valvular insufficiency or regurgitation occurs when the valve does not close completely, allowing blood to flow backwards, thereby causing the heart to be less efficient. A diseased or damaged valve, which can be congenital, age-related, drug-induced, or in some instances, caused by infection, can result in an enlarged, thickened heart that loses elasticity and efficiency. Some symptoms of heart valve diseases can include weakness, shortness of breath, dizziness, fainting, palpitations, anemia and edema, and blood clots which can increase the likelihood of stroke or pulmonary embolism. Symptoms can often be severe enough to be debilitating and/or life threatening.

Heart valve prostheses have been developed for repair and replacement of diseased and/or damaged heart valves. Such heart valve prostheses can be percutaneously delivered and deployed at the site of the diseased heart valve through catheter-based delivery systems. Such heart valve prostheses are delivered in a radially compressed or crimped configuration so that the heart valve prosthesis can be advanced through the patient's vasculature. Once positioned at the treatment site, the heart valve prosthesis is expanded to engage tissue at the diseased heart valve region to, for instance, hold the heart valve prosthesis in position.

While these valve prostheses offer minimally invasive methods for heart valve repair and/or replacement, challenges remain when trying to provide valve prosthesis to a target location using smaller catheter sizes. For example, when implanting a mitral valve prosthesis in a native mitral valve, it is important that the left ventricular outflow tract (LVOT) is not impinged, blocked, or obstructed. Therefore, an interest exists in ensuring that, as valve prostheses decrease in size, blood flow via the LVOT is not obstructed during systole.

The present disclosure relates to improvements in a heart valve prosthesis to ensure that the heart valve prosthesis has a low profile for transcatheter delivery through a patient's vasculature.

### SUMMARY

According to a first embodiment hereof, the present disclosure provides a heart valve prosthesis including a frame having an inner portion, an outer portion, and a skirt. The inner portion is configured to support a prosthetic valve component. The outer portion is coupled to the inner portion, the outer portion being sized to surround the inner portion and configured to anchor the prosthesis. The outer portion including a plurality of first crowns positioned around a first end of the outer portion and a plurality of second crowns positioned inward of the plurality of first crowns. The skirt is disposed within and coupled to the outer portion, wherein an end segment of the skirt is disposed proximate to the first end of the outer portion. The end segment of the skirt does not substantially extend past the plurality of second crowns of the outer portion so as to leave a substantially unobstructed blood flow passageway through each of the pair of adjoining crowns.

In an aspect of the first embodiment, and in combination with any other aspects herein, the disclosure provides that the outer portion includes a plurality of Y-shaped struts, each coupled to one of the plurality of first crowns and positioned proximate the first end.

In an aspect of the first embodiment, and in combination with any other aspects herein, the disclosure provides that each of the Y-shaped struts includes a node, a primary strut that extends between a respective first crown of the plurality of first crowns and the node, and a pair of secondary struts branching, at the node, from the primary strut toward a second end of the outer portion.

In an aspect of the first embodiment, and in combination with any other aspects herein, the disclosure provides that the skirt is coupled to each Y-shaped strut and each second crown by a suture.

In an aspect of the first embodiment, and in combination with any other aspects herein, the disclosure provides that the skirt is coupled to the primary strut and each of the pair of secondary struts of each Y-Shaped strut.

In an aspect of the first embodiment, and in combination with any other aspects herein, the disclosure provides that a first line of stitches couples the skirt to a first portion of each Y-shaped strut and a second line of stitches couples the skirt to a second portion of each Y-shaped strut.

In an aspect of the first embodiment, and in combination with any other aspects herein, the disclosure provides that each of the first line of stitches and the second line of stitches forms a repeating pattern of axial segments and circumferential segments.

In an aspect of the first embodiment, and in combination with any other aspects herein, the disclosure provides that the first line of stitches and the second line of stitches are completed with a single continuous suture.

According to a second embodiment herein, the present disclosure provides a method for manufacturing a heart valve prosthesis. The method including attaching an inner skirt to an inner portion and attaching an outer skirt to an outer portion and coupling the inner portion to the outer portion. A plurality of pairs of adjoining crowns are defined at the points where the inner portion and the outer portion are coupled. Further, an end segment of the outer skirt, disposed proximate to a first end of the outer portion, does not substantially extend past a plurality of second crowns of the outer portion as to leave a substantially unobstructed blood flow passageway through each pair of adjoining crowns.

In an aspect of the second embodiment, and in combination with any other aspects herein, the disclosure provides that the outer portion has a stent-like structure with a second end opposite the first, the outer portion including a plurality of first crowns, a plurality of second crowns, and plurality of Y-shaped strut positioned at the first end.

In an aspect of the second embodiment, and in combination with any other aspects herein, the disclosure provides that each of the Y-shaped struts includes a node, a primary strut is positioned between a respective first crown of the plurality of first crowns and the node, and a pair of secondary struts branching, at the node, from the primary strut toward the second end of the outer portion.

In an aspect of the second embodiment, and in combination with any other aspects herein, the disclosure provides that the outer skirt is coupled to each Y-shaped struts and each second crown by a suture.

In an aspect of the second embodiment, and in combination with any other aspects herein, the disclosure provides that the outer skirt is coupled to the primary strut and each of the pair of secondary struts of each Y-Shaped strut.

In an aspect of the second embodiment, and in combination with any other aspects herein, the disclosure provides a first line of stitches couples the skirt to a first portion of each Y-shaped strut and a second line of stitches couples the skirt to a second portion of each Y-shaped strut.

In an aspect of the second embodiment, and in combination with any other aspects herein, the disclosure provides that each of the first line of stitches and the second line of stitches forms a repeating pattern of axial segments and circumferential segments.

In an aspect of the second embodiment, and in combination with any other aspects herein, the disclosure provides that the first line of stitches and the second line of stitches are completed with a single continuous suture.

### BRIEF DESCRIPTION OF DRAWINGS

The foregoing and other features and advantages of the invention will be apparent from the following description of embodiments thereof as illustrated in the accompanying drawings. The accompanying drawings, which are incorporated herein and form a part of the specification, further serve to explain the principles of the invention and to enable a person skilled in the art to make and use the invention. The drawings are not to scale.

FIG. 1 depicts a perspective view of a heart valve prosthesis in accordance with an aspect of this disclosure.

FIG. 2 depicts a ventricle, or bottom, outflow view of the heart valve prosthesis of FIG. 1 in accordance with an aspect of this disclosure.

FIG. 3 depicts a perspective view of an inner portion of the heart valve prosthesis of FIG. 1 in accordance with an aspect of this disclosure.

FIG. 4 depicts a perspective view of an outer portion of the heart valve prosthesis of FIG. 1 in accordance with an aspect of this disclosure.

FIG. 5 depicts a perspective view of an outflow end of the heart valve prosthesis of FIG. 1 in accordance with an aspect of this disclosure.

FIG. 6 depicts an expanded view of an area A of the heart valve prosthesis of FIG. 5 in accordance with an aspect of this disclosure.

FIG. 7 depicts a schematic view of the heart valve prosthesis of FIG. 1 positioned within a heart.

FIG. 8 depicts a ventricle, or bottom, outflow view of the outer portion of the heart valve prosthesis of FIG. 1 in accordance with an aspect of this disclosure.

FIG. 9 depicts an atrial, or top, inflow view of the outer portion of the heart valve prosthesis of FIG. 1 in accordance with an aspect of this disclosure.

FIG. 10A depicts, for illustrative purposes only, a cut and flattened outer portion and outer skirt of the heart valve prosthesis of FIG. 1 showing a first line of stitches of a stitching pattern in accordance with an aspect of this disclosure.

FIG. 10B depicts, for illustrative purposes only, a cut and flattened outer portion and outer skirt of the heart valve prosthesis of FIG. 1 showing a second line of stitches of the stitching pattern in accordance with an aspect of this disclosure.

FIG. 10C depicts, for illustrative purposes only, a cut and flattened outer portion and outer skirt of the heart valve prosthesis of FIG. 1 showing the completed stitching pattern, with the first line of stitches shown in FIG. 10A and the second line of stitches shown in FIG. 10B, in accordance with an aspect of this disclosure.

### DETAILED DESCRIPTION

Specific embodiments of the present invention are now described with reference to the figures, wherein like reference numbers indicate identical or functionally similar elements.

Specific embodiments of the present invention are now described with reference to the figures. The terms “inflow” and “outflow”, when used in the following description refer to a native vessel, native valve, or a device to be implanted into a native vessel or native valve, such as a heart valve prosthesis, are with reference to the direction of blood flow. Thus, “inflow” refers to positions in an upstream direction with respect to the direction of blood flow and the term “outflow” refers to positions in a downstream direction with respect to the direction of blood flow.

The following detailed description is merely exemplary in nature and is not intended to limit the invention or the application and uses of the invention. Although the description of embodiments hereof is in the context of the treatment of heart valves such as the pulmonary, aortic, mitral, or tricuspid valve, the invention may also be used in other body passageways where it is deemed useful. Furthermore, there is no intention to be bound by any expressed or implied theory presented in the preceding technical field, background, brief summary or the following detailed description.

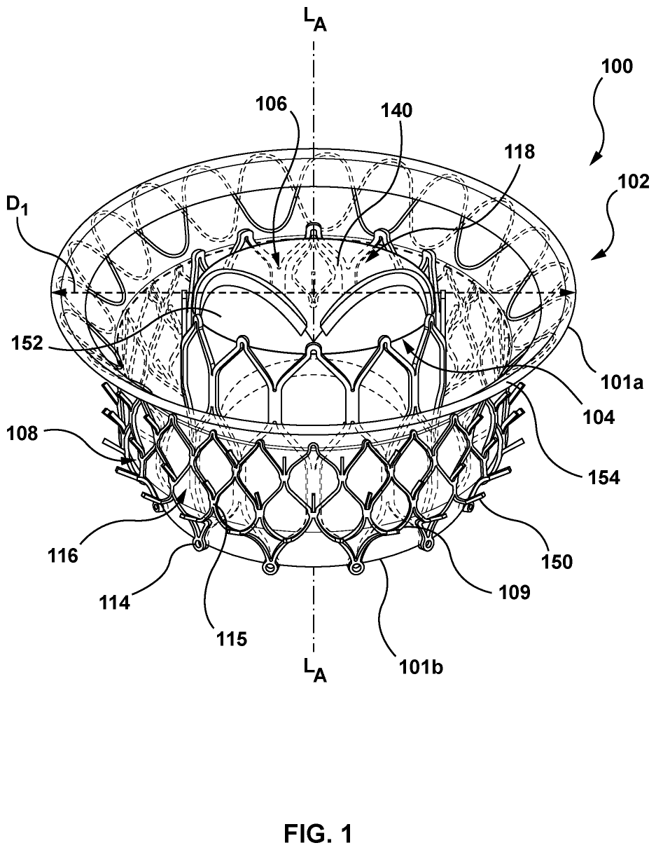

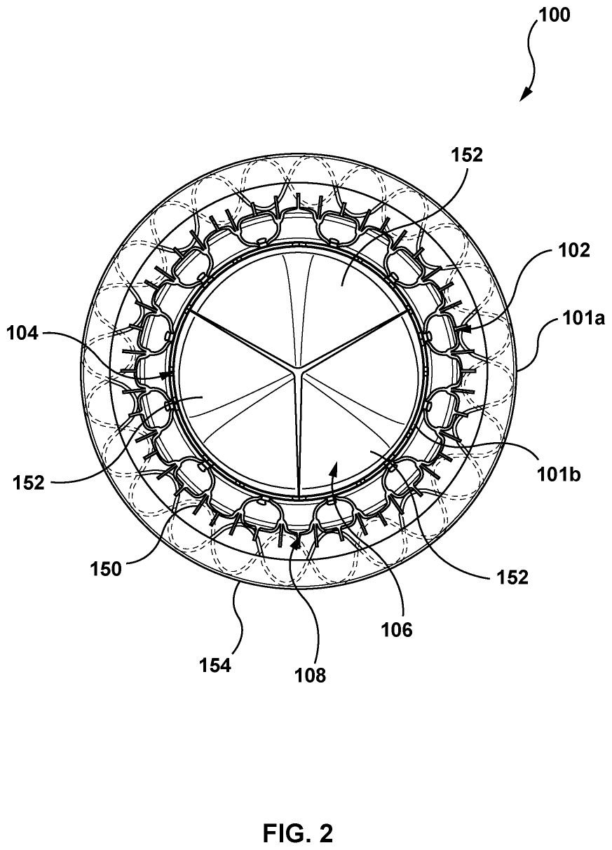

A perspective view of a transcatheter heart valve prosthesis 100 in accordance with an aspect of the disclosure is shown in FIG. 1, with FIG. 2 depicting a bottom or outflow view of the heart valve prosthesis 100. The heart valve prosthesis 100 is configured to be compressed into a reduced-diameter delivery configuration within a delivery catheter and to return to an expanded, deployed configuration when delivered/released from the delivery catheter within a native mitral valve. In some embodiments, the heart valve prosthesis 100 includes a frame 102 and a prosthetic valve component 106. The frame 102 has a stent-like structure that is configured to support the prosthetic valve component 106 and to define, along a longitudinal axis L_{A} thereof, a blood flow lumen 140 that substantially extends from an inflow end 101a to an outflow end 101b of the heart valve prosthesis 100. In some embodiments, the frame 102 generally includes an inner portion 104 and an outer portion 108. In aspects hereof, the inner portion 104 of the frame 102 may be alternatively referred to as a valve support element, an inner frame, or valve housing, and/or the outer portion 108 of the frame 102 may be alternatively referred to as an anchoring element, fixation ring, or an outer frame. The inner portion 104 is configured to hold the prosthetic valve component 106, and the outer portion 108, which surrounds the inner portion 104, is configured to secure the heart valve prosthesis 100 to the native tissue of the heart when implanted in vivo. The frame 102 may be considered to have a dual-stent structure, i.e., an inner stent and an outer stent.

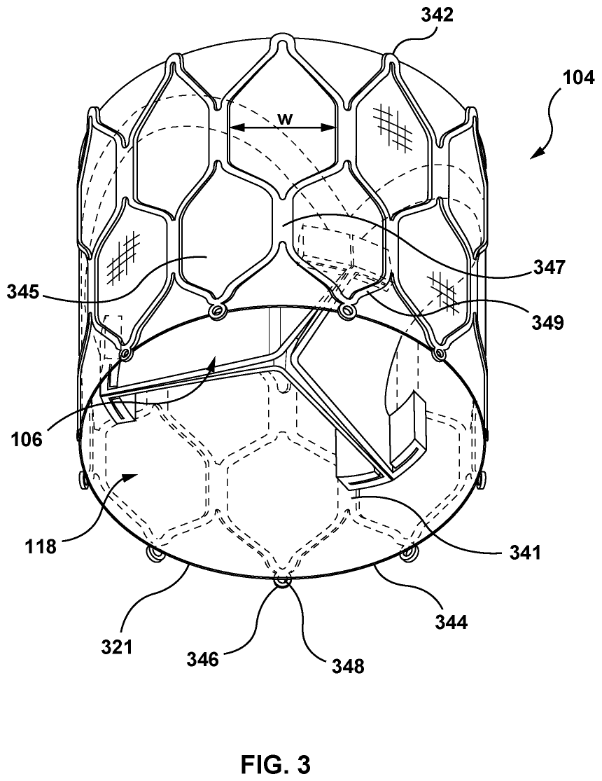

The inner portion 104 is positioned within the outer portion 108 so as to be spaced therefrom. FIG. 3 depicts a perspective view of the inner portion 104 of the heart valve prosthesis 100 in accordance with an aspect of this disclosure and shows the interior of an outflow end 344 of the inner portion 104. The inner portion 104 generally forms a hollow cylindrical shape having a substantially constant diameter from an inflow end 342 to the outflow end 344 thereof. The stent-like structure of the inner portion 104 defines a plurality of open cells 345 arranged in a honeycomb pattern. Further, the inner portion 104 has a plurality of crowns 346 positioned at the inflow end 342 and the outflow end 344 and, in some embodiments, the crowns 346 at the outflow end 344 may have an aperture 348 that allows for the inner portion 104 to be coupled to the outer portion 108 as described in detail below.

Each cell 345 of the inner portion 104 is defined by a series of struts 349, with one end of each cell 345 being defined by an endmost crown 346 and the other end of each cell 345 being defined by a node 347, with the node 347 being a thicker strut segment formed between adjacent cells 345. The node 347 may be defined as a connection point of various struts 349. The inner portion 104 is shown in the embodiment of FIG. 3 as having two rows of cells 345, however, this is merely exemplary and additional rows of cells 345 or patterns of cells 345 is envisioned without departing from the scope of the present disclosure.

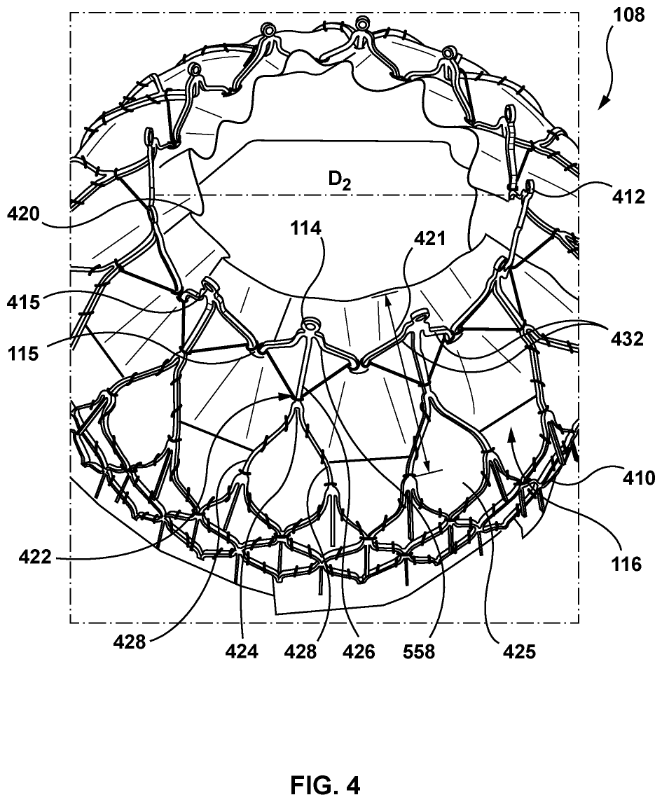

The outer portion 108 is configured to secure the heart valve prosthesis 100 to the native valve and the surrounding subannular tissue, such as the inward facing-surface of the leaflets A perspective view of the outer portion 108, from its outflow end 412, is shown in FIG. 4. The outer portion 108 is positioned around the inner portion 104 and defines an inflow end 410 having a first diameter D1 and the outflow end 412 having a second diameter D2 that is smaller than the first diameter D1. A transition portion 109 is positioned between the inflow end 410 and the outflow end 412 of the outer portion 108. As shown in FIG. 4, the transition portion 109 reduces in diameter between the inflow end 410 and the outflow end 412, acting as a taper between the first diameter D1 and the second, smaller diameter D2. At least a portion of the outer surface of the outer portion 108, when the heart valve prosthesis 100 is in an expanded state, is configured to be disposed against the native tissue of the heart for securing the outer portion 108 and, concurrently, the heart valve prosthesis 100 at the implantation site. Further, the outer portion 108 is spaced apart from the inner portion 104. In more detail, the outer portion 108 may deform upon implantation within a native mitral valve annulus, and/or expand and contract in response to movement of the native tissue during diastole and systole, while remaining spaced from the inner portion 104, which thereby permits the inner portion 104 to remain relatively still and undeformed. The inner portion 104 is, therefore, somewhat protected from external forces such that deformation from the outer frame is reduced/dampened, allowing for the prosthetic valve component 106 to more efficiently replicate the function of the native mitral valve. In addition, the outer portion 108 may further include a plurality of prongs 150 that extend radially from the outer surface of the outer portion 108 and are configured to engage with the native tissue, further fixating the outer portion 108 to the native tissue.

To further explain the stent-like or lattice structure of the outer portion 108, a plurality of cells 425 are defined across the surface of the outer portion 108. At the outflow end 412 of the outer portion 108 a plurality of first crowns 114 and a plurality of second crowns 115 are formed by respective pairs of opposing struts 432. Each first crown 114 is circumferentially spaced from an adjacent first crown 114 by a second crown 115. The plurality of second crowns 115 are spaced inwardly, or toward the inflow end 410, of the plurality of first crowns 114 with struts 432 extending therebetween. The pluralities of first and second crowns 114, 115 with struts 432 therebetween form a substantially wavy or zig- zag patterned ring at the outflow end 412 of the outer portion 108 that is sized and configured to substantially correspond to the pattern of crowns 346, nodes 347, and struts 349 at the outflow end 344 of the inner portion 104. Further, the first crowns 114 may be coupled to a plurality of Y-shaped struts 422 that make-up the transition portion 109 and provide a transition from the first diameter D1 to the second diameter D2 by bending inward towards the longitudinal axis L_{A}. In alternative embodiments, it is envisioned that the plurality of Y-shaped struts 422 may make up a portion or the entirety of the transition portion 109. In some embodiments, there is an equivalent number of second crowns 115 as there are first crowns 114. Further, each first crown 114 of the outer portion 108 has an aperture 415 that allows for the outer portion 108 to be coupled or connected to a corresponding crown 346 of the inner portion 104.

To explain further, the Y-shaped struts 422 may include a primary strut 426 that is connected to a pair of secondary struts 428 at a node 424. The pair of secondary struts 428 are coupled to the node 424 at one end of the secondary struts 428 and may form a portion of the transition portion 109. Alternatively, the secondary struts 428 may be angled as to act as the transition portion 109, with the secondary struts 428 assisting in the transition from the first diameter D1 to the second diameter D2. Further, each primary strut 426 may be coupled at one end to one of the plurality of first crowns 114.

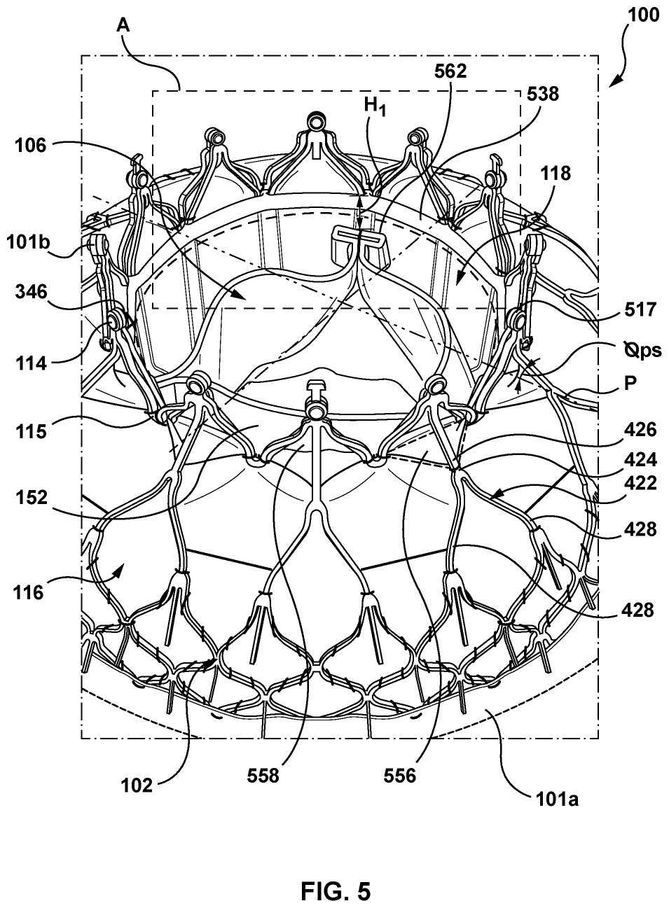

An angle θ_{PS} of the primary strut 426 is defined as the angle between the primary strut 426 and a plane P that is parallel to the outflow end 412 of the outer portion 108, wherein the plane P is positioned at the axial position of the node 424. Plane P is shown in FIG. 5. In accordance with aspects hereof, the angle θ_{PS} may be increased or decreased as to manage the forces placed on the Y-shaped strut 422 and the plurality of first crowns 114. For example, as the angle is increased, potential strain placed on the Y-shaped strut 422 is better distributed along the length of the Y-shaped strut 422, and the increased angle also assists in mitigating or reducing the transmission of deformations from the outer portion 108 to the inner portion 104. Further, an increase in the angle of the primary strut 426 reduces the amount of potential blockage of blood flow from the Left Ventricle (LV) to the Left Ventricular Outflow Tract (LVOT). In some embodiments, wherein the valve prosthesis 100 must be crimped prior to deployment, the increased angle of the primary strut 426 reduces the required force to crimp the transcatheter valve prosthesis 100 to a smaller diameter.

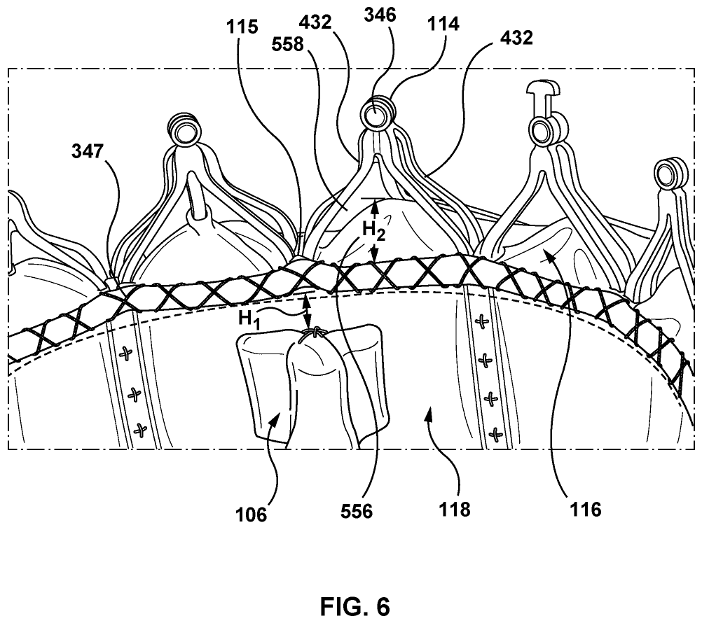

As described above, the plurality of crowns 346 at the outflow end 344 of the inner portion 104 and the plurality of first crowns 114 at the outflow end 412 of the outer portion 108 have apertures 348, 415 that algin with one and other to permit the coupling together of the inner portion 104 and the outer portion 108. When one of the crowns 346 of the inner portion 104 is coupled to one of the first crowns 114 of the outer portion 108, a pair of adjoining crowns 517 is formed and, depending on the configuration of the inner portion 104 and the outer portion 108, the number of pairs of adjoining crowns 517 may vary. Further, the pairs of adjoining crowns 517 may be joined by rivets, welding, or other methods known in the art. The pairs of adjoining crowns 517 are shown in FIG. 5, with FIG. 5 being a perspective view of the heart valve prosthesis 100 from the outflow end 101b and FIG. 6 being an enlarged view of an area A of FIG. 5. The heart valve prosthesis 100 is shown in embodiments hereof as having twelve pairs of adjoining crowns 517, however, this is merely an exemplary heart valve prosthesis 100 and configurations of the heart valve prosthesis 100 are envisioned that may include other numbers of pairs of adjoining crowns 517.

In accordance with aspects hereof, the inner portion 104 and the outer portion 108 of the frame 108 of the heart valve prothesis 100 may be made from any number of suitable biocompatible materials, e.g., stainless steel, nickel titanium alloys such as Nitinol™, cobalt chromium alloys such as MP35N, other alloys such as ELGILOY® (Elgin, Ill.), various polymers, pyrolytic carbon, silicone, polytetrafluoroethylene (PTFE), or any number of other materials or combination of materials. A suitable biocompatible material would be selected such that the heart valve prosthesis 100 may be configured to be compressed into a reduced diameter configuration for transcatheter delivery to a native valve, whereby release from a delivery catheter allows the prosthesis 100 to self-expand, returning to an expanded, deployed configuration. In some embodiments, the self-expansion is accomplished through the use of a shape-memory material such as Nitinol™. The heart valve prosthesis 100 may be processed to have a default or “set” shape that coincides with the deployed configuration. Therefore, once the compressed heart valve prosthesis 100 is delivered and released, the prosthesis 100 will return to the default or “set” deployed configuration.

The prosthetic valve component 106 of the heart valve prosthesis 100 is capable of regulating blood flow therethrough via valve leaflets 152. FIGS. 1-3, 5, and 6 illustrate an exemplary prosthetic valve component 106 having three leaflets 152, although a bicuspid or two leaflet configuration may alternatively be used in embodiments hereof. When deployed in situ, the prosthetic valve component 106 in a closed state is configured to block blood flow in one direction to regulate blood flow through the blood flow lumen 140 of the inner portion 104. The valve leaflets 152 are disposed to coapt within the inner portion 104 and may be secured or otherwise fused to the inner portion 104, such that the valve leaflets 152 open during diastole. The leaflets 152 are attached along their bases to the inner portion 104, for example, using sutures or a suitable biocompatible adhesive. Adjoining pairs of leaflets 152 are attached to one another at their lateral ends to form leaflet commissures 562. The orientation of the leaflets 152 within the inner portion 104 depends upon which end of the heart valve prosthesis 100 is the inflow end 101ₐ and which end of the heart valve prosthesis 100 is the outflow end 101b, thereby ensuring one-way flow of blood through the heart valve prosthesis 100. As shown in FIG. 6, the prosthetic valve component 106 is operably coupled to the inner portion 104 at a distance H₁ inwardly from the nodes 347. The distance H₁ is the measured axial displacement between the commissures 562 of the prosthetic valve component 106 and the nodes 347 of the inner portion 104. By displacing the prosthetic valve component 106 the distance H₁, the prosthetic valve component 106 is encompassed inside the inner skirt 118, thereby protecting the prosthetic valve component 106 from being pinched, cut, or otherwise damaged. Further, the distance H₁ can be optimized to the configuration and design of the frame 102 as to avoid or reduce the risk of damage to the prosthetic valve component 106.

Further, the leaflets 152 may be formed of various flexible materials including, but not limited to, natural pericardial material such as tissue from bovine, equine or porcine origins, or synthetic materials such as polytetrafluoroethylene (PTFE), DACRON® polyester, pyrolytic carbon, or other biocompatible materials. With certain prosthetic leaflet materials, it may be desirable to coat one or both sides of the replacement valve leaflet with a material that will prevent or minimize overgrowth. It is further desirable that the prosthetic leaflet material is durable and not subject to failure due to stretching, deforming, or fatigue.

The heart valve prosthesis 100 may further include a brim or preshaped wire element 154 that extends outwardly from the inflow end 410 of the outer portion 108. The brim 154 includes overlapping, 180 degree out of phase, sinusoidal wire forms attached to a suitable biocompatible low-profile fabric, with the fabric being hinged to the outer portion 108. A suitable biocompatible low-profile fabric may be one used in bioprosthetic implants, namely, endovascular grafts, valves or left atrial appendage devices, to promote bio-integration, such as woven polyethylene terephthalate (PET) fabric. The brim element 154 may act as an atrial retainer, if present, and to serve such a function the brim element 154 may be configured to engage tissue above a native annulus, such as a supra-annular surface or some other tissue in the left atrium, to thereby inhibit downstream migration of the heart valve prosthesis 100 as well as mitigate leakage through any gaps between native tissue and the brim, for e.g., during atrial systole.

An inner skirt 118 is disposed within and is coupled to the inner portion 104, and, more particularly, is coupled as to line an inner surface 341 of the inner portion 104, or at least a substantial portion thereof. In an aspect hereof, the inner skirt 118 extends from the inflow end 342 of the inner portion 104 to the outflow end 344 of the inner portion 104, with an outflow edge 321 of the inner skirt 118 being defined at the outflow end 344 of the inner portion 104. When the heart valve prothesis 100 is implanted within a native mitral valve, the inner skirt 118 is configured to limit the amount of unintentional blood leakage, otherwise known as regurgitation, between a left atrium and a left ventricle. In further detail, the inner skirt 118 covers the inner surface 341 of the inner portion 104, as to only allow blood to flow from the atrium to the ventricle when the prosthetic valve component 106 is in the open state. The inner skirt 118 may take the form of a single piece or multiple pieces of material that is wrapped within the inner surface 341 as to create a cylindrical body that is flush with the inner surface 341. The inner skirt 118 is then affixed to the inner portion 104 using sutures or adhesive. In order to inhibit blood flow, the inner skirt 118 is further configured to substantially cover the cells 345 of the inner portion 104.

Similarly, an outer skirt 116 is disposed within and is coupled to the outer portion 108. In more detail, the outer skirt 116 extends from the inflow end 410 of the outer portion 108 toward the outflow end 412 of the outer portion 108. The outer skirt 116 includes an outflow end 420 which is integrated within a skirt seal 538, which is discussed in further detail below. As discussed previously, the outer portion 108 has the first diameter D1 at the inflow end 410 that is larger than the second diameter D2 at the outflow end 412, therefore, the outer skirt 116 is configured to match the tapered profile of the transition portion 109. When the heart valve prothesis 100 is implanted within a native mitral valve, the outer skirt 116 substantially covers the inner surface of the outer portion 108 so as to limit unintentional blood flow from the left atrium to the left ventricle. The outer skirt 116 may take the form of a single piece or multiple pieces of material that is wrapped within the inner surface as to create a shape that is flush with the outer portion 108. The outer skirt 116 is then affixed to the outer portion 108 using sutures or adhesive. In some embodiments, the outer skirt 116 may be sewn onto the outer portion 108 using a series of sutures. In order to inhibit blood flow, the outer skirt 116 is further configured to substantially cover the cells 425 of the outer portion 108. In an aspect of the invention, the combination of the outer skirt 116 and the inner skirt 118 permits blood to circulate or recirculate in the open area or trough created between the inner portion 104 and the outer portion 108, and it has been found that after implantation, and over time, slowly tissue ingrowth covers the whole open (trough) area to thereby bio-integrate the heart valve prothesis 100 into the mitral environment.

In further detail, the outer skirt 116 may be coupled to the plurality of Y-shaped struts 422 at each of the primary struts 426 in order to prevent the outer skirt 116 from sliding axially towards the outflow end 412 of the outer portion 108. The primary struts 426 act as a series of anchoring points to secure the outer skirt 116 to the outer portion 108, reducing potential motion of the outer skirt 116, and thereby improving the long term durability of the outer skirt 116. As shown in FIG. 5, denoted by dashed lines, a tent 556 is formed by a bulging, bunching, or protrusion of material within an end segment 421 of the outer skirt 116 that extends outward axially relative to the second crowns 115. The end segment 421 of the outer skirt 116 may extend axially from the transition portion 109 to the outflow end 412 of the outer portion 108. A plurality of tents 556 are further shown in FIG. 6 and are each positioned inward of the first crowns 114 and outward of the second crowns 115 and between the opposing struts 432 that make up each first crown 114. An axial distance between a respective second crown 115 and the top of a respective tent 556 is defined as H₂. The tent 556 is created as a result of attaching the outer skirt 116 to the primary strut 426 and the tent height H₂ may be controlled by how and where along the length of the primary strut 426 the outer skirt 116 is attached. For example, connecting the outer skirt 116 to the primary strut 426 at or closer to the first crown 114 will result in a larger tent height H₂, while connecting the outer skirt 116 closer to the node 424 will result in a smaller tent height H₂. Further, in some embodiments, there are as many tents 556 as there are first crowns 114 and second crowns 115.

The inner skirt 118 is coupled to the inner portion 104 and the outer skirt 116 is coupled to the outer portion 108 prior to the inner portion 104 and the outer portion 108 being coupled to one another. In some embodiments, after assembly of the inner portion 104 within the outer portion 108, the outflow end 344 of the inner skirt 118 and the outflow end 412 of the outer skirt 116 are coupled together using a skirt seal 538 in order to limit unintentional blood leakage, as described in co-pending application U.S. Patent Appl. No. 63/251,338 that is incorporated by reference herein in its entirety and for all purposes. Therefore, when the inner portion 104 is coupled to the outer portion 108, the outer skirt 116 is positioned outside of the inner portion 104 and, subsequently, is pinched between the nodes 347 of the inner portion 104 and the second crowns 115 of the outer portion 108.

In aspects hereof, the inner skirt 118 and the outer skirt 116 may be formed of a low-porosity woven fabric, such as polyester, Dacron fabric, or PTFE. In further aspect, the inner and outer skirts 118, 116 may be a knit polyester, such as a polyester or PTFE knit, which can be used when it is desired to provide a medium for tissue ingrowth and the ability for the fabric to stretch to conform to a curved surface. Polyester velour fabrics may alternatively be used, such as when it is desired to provide a medium for tissue ingrowth on one side and a smooth surface on the other side. These and other appropriate cardiovascular fabrics are commercially available from Bard Peripheral Vascular, Inc. of Tempe, Ariz., for example. In alternative aspects hereof, the inner and outer skirts 118, 116 may be formed of a natural or biological material such as pericardium or another membranous tissue such as intestinal submucosa. Further, it is envisioned that the inner skirt 118 and the outer skirt 116 may be made of the same or different materials, for example, the inner skirt 118 may be made from a PTFE knit, while the outer skirt 116 is made of a woven polyester. It is further envisioned, that in some embodiments, it may be beneficial to have skirts of varying thicknesses, such as, an inner skirt 118 that is thicker than an outer skirt 116.

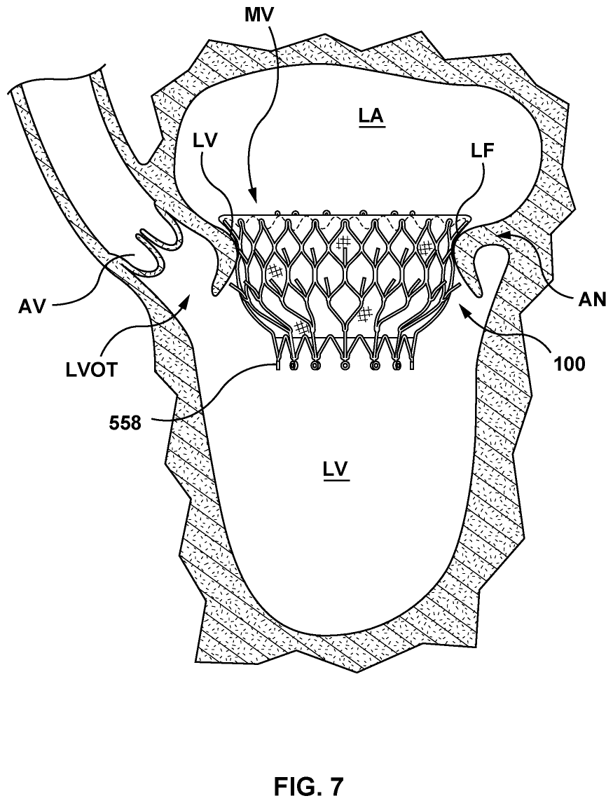

FIG. 7 is a schematic sectional illustration of a heart that depicts a left ventricle LV, a left atria LA, a left ventricular outflow tract LVOT, an aortic valve AV, a mitral valve MV, and a mitral valve annulus AN. The left atrium LA pumps the oxygenated blood through the mitral valve MV and into the left ventricle LV during ventricular diastole. The left ventricle LV contracts during systole and blood flows outwardly from the left ventricle LV through the left ventricle outflow tract LVOT and into the aorta via the aortic valve AV and to the remainder of the body. The valve leaflets LF attach the surrounding heart structure via a dense fibrous ring of connective tissue called an annulus AN which is distinct from both the leaflet tissue LF as well as the adjoining muscular tissue of the heart wall. In general, the connective tissue at the annulus AN is more fibrous, tougher and stronger than leaflet tissue. In some embodiments the heart valve prosthesis 100 is positioned within the annulus AN, pressing the valve leaflets LF of the mitral valve against the walls of the left ventricle LV. The deployment of the heart valve prosthesis 100 within the native mitral valve MV may create a risk that portions of the heart valve prosthesis 100 will block or interrupt blood flow out of the left ventricle LV, i.e., through the left ventricular outflow tract LVOT, to the aortic valve AV, increasing the risk of serious complications such as blood thrombus.

In order to avoid obstruction of the LVOT by a heart valve prosthesis 100 in accordance herewith, in an aspect of the disclosure a plurality of blood flow passageways 558 are created or maintained through the outflow end 101b of the heart valve prosthesis 100. Each blood flow passageway 558 may be defined as an unobstructed area through a pair of adjoining crowns 517 that permits blood to flow therethrough. With each aforementioned tent 556 being positioned beneath a pair of adjoining crowns 517, within a single blood flow passageway 558, the tents 556 must be sized so as to avoid partially or completely blocking the passage of blood through the blood flow passageway 558. As stated above, the blocking of blood flow may reduce the effective cardiac output through the aortic valve, as well as lead to an increased risk of serious health complications, therefore, limiting or minimizing the tent height H₂ can reduce the risk of such complications. For example, if the blood flow passageways 558 are obstructed by the tents 556, blood could begin to stagnate at the outflow end 101b of the heart valve prosthesis 100, increasing the risk of the formation of thrombi on the heart valve prosthesis 100. Limiting the tent height H₂ may be accomplished by either flattening the angle of the primary struts 426 relative to the first crowns 114 and/or by coupling the outer skirt 116 closer to the node 424. The blood flow passageway 558 may be considered substantially unobstructed when there is sufficient blood flow through the passageway as to minimize the ability for blood to stagnate around the outflow end 101b of the heart valve prosthesis 100. In more detail, substantially unobstructed may mean that there is no obstruction of a cross-section to a blood flow passageway 558, that no more than 10 percent of a cross-section of a blood flow passageway 558 is obstructed, no more than 20 percent of a cross-section of a blood flow passageway 558 is obstructed, no more than 30 percent of a cross-section of a blood flow passageway 558 is obstructed, no more than 40 percent of a cross-section of a blood flow passageway 558 is obstructed, no more than 50 percent of a cross-section of a blood flow passageway 558 is obstructed, no more than 60 percent of a cross-section of a blood flow passageway 558, or any percent in between 0 percent and 60 percent. In some embodiments, the tent height H₂ is configured to allow 50% or more of each blood flow passageway 558 to be open or unobstructed, as to allow sufficient blood flow to reduce the risk of stagnation.

In some embodiments, in order to achieve a lower profile heart valve prosthesis 100, it is favorable to have a steeper angled primary strut 426, therefore, to reduce the tent height H₂, the end segment of 421 of the outer skirt 116 may be coupled to the end of each primary strut 426 closest to the nodes 424. In particular, the end segment 421 of the outer skirt 116 may be coupled to the Y-shaped struts 422 at the primary struts 426 and at both the secondary struts 428. The end segment 421 of the outer skirt 116 may further be attached to each of the second crowns 115.

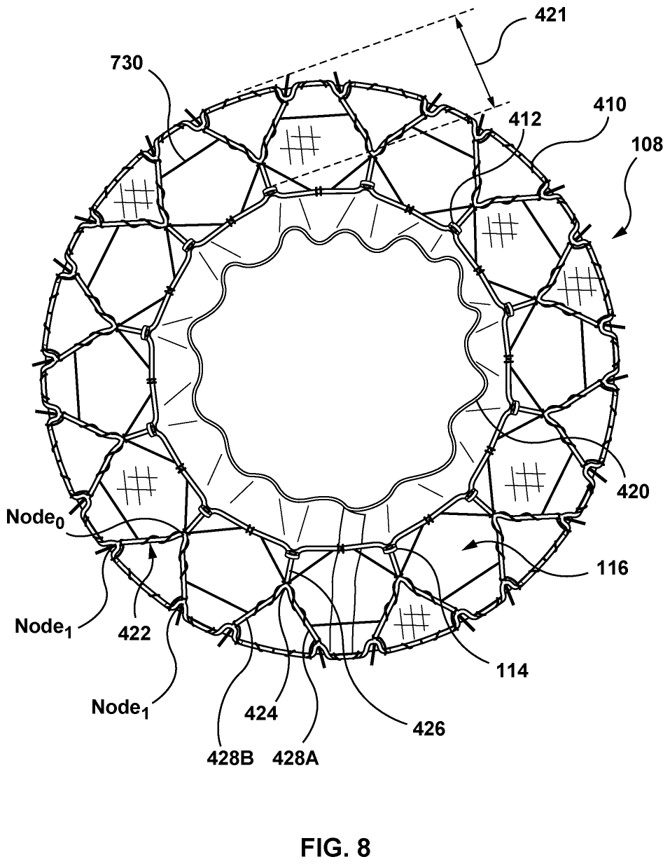

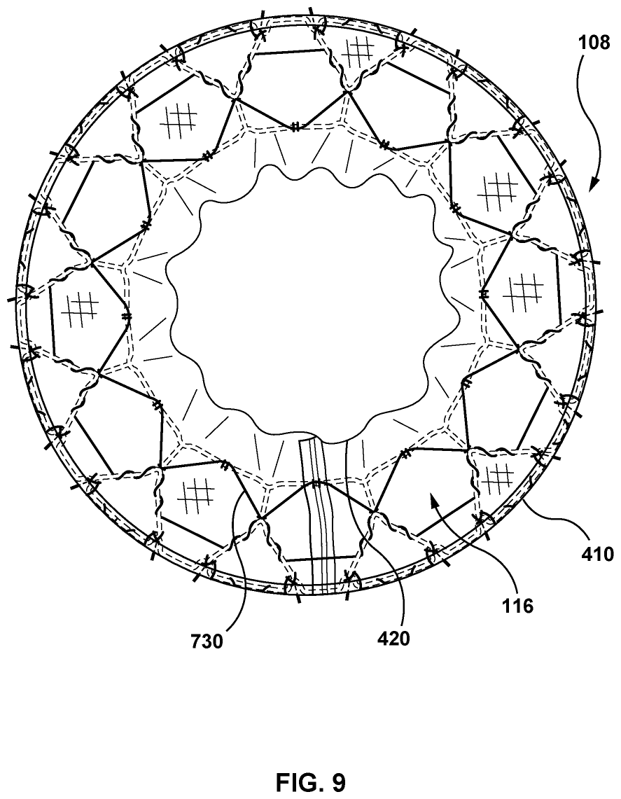

In an aspect hereof a stitching pattern shown in FIGS. 8 and 9 may be used to reduce a tent height H₂, wherein FIG. 8 shows the outer portion 108 from a ventricle side, or a bottom, and FIG. 9 shows the outer portion 108 from an atrial side, or a top. The stitching pattern described herein, in some embodiments, may reduce the tent height by 2 mm-2.5 mm in comparison to stitching patterns known in the art. Further, the stitching pattern described hereafter, allows for a single stitch to be placed at the node 424 of the Y-shaped strut 422 or on the primary strut 426. By using only a singular suture to couple the outer skirt 116 to each of the Y-shaped struts 422, the tent height H₂ may be reduced as much as possible, while ensuring that the end segment 420 of the outer skirt 116 does not slip or slide axially towards the outflow end 101b. It should be understood by the description of a stitching pattern in aspects hereof that the remainder of the outer skirt 116 is attached to the remainder of the outer portion 108 using one or more other sutures and/or stitching pattern(s).

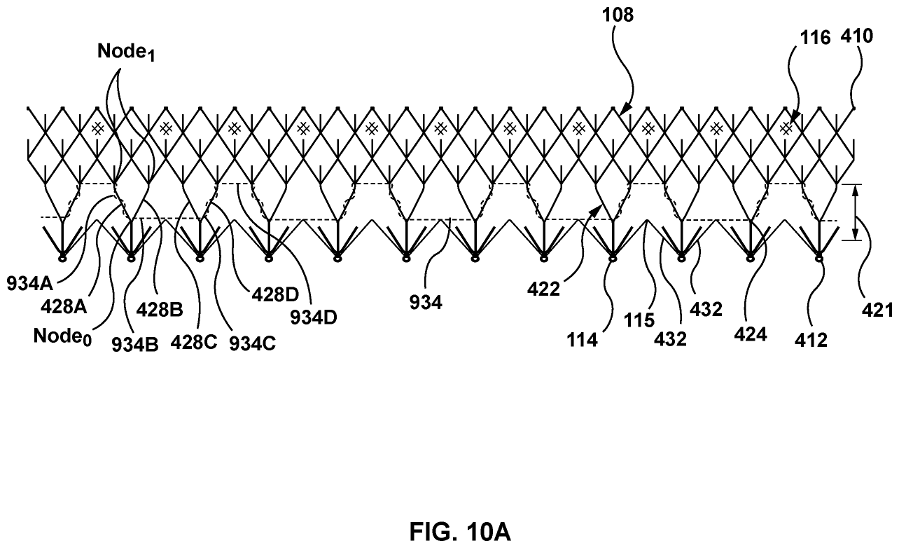

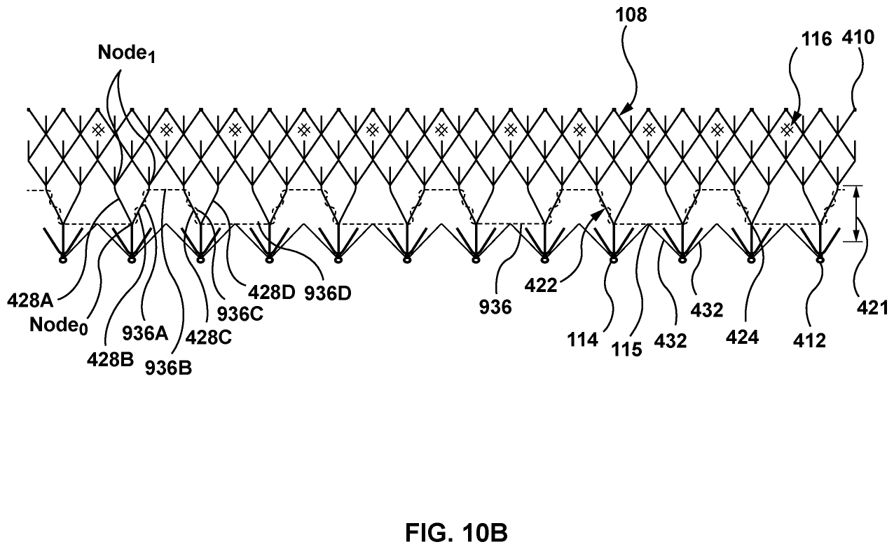

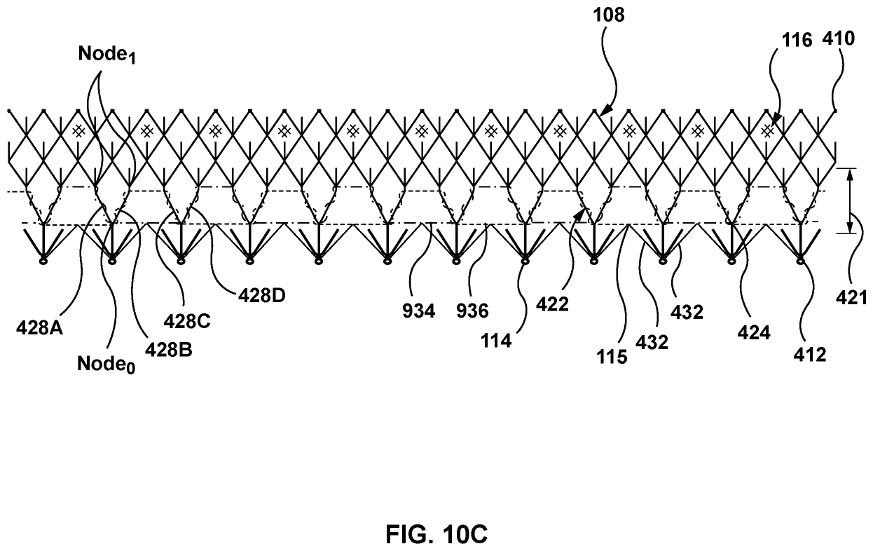

Further, the stitching pattern, as shown in FIGS. 8 and 9, does not allow for the end segment 420 of the outer skirt 116 to substantially extend past the plurality of second crowns 115 of the outer portion 412 so as to leave the blood flow passageway 558 substantially unobstructed as described above. This stitching pattern is also shown in FIGS. 10A, 10B, and 10C which depict, for illustrative purposes only, a cut and flattened outer portion 108 of the frame 102 with the outer skirt 116 attached thereto and removed from a remainder of the heart valve prosthesis 100 in accordance with an aspect of this disclosure. For clarity, the node 424 where each secondary strut 428 is coupled to the primary strut 426 is denoted as Node₀, and the position where an opposing end of each secondary strut 428 is coupled to the remaining outer portion is denoted as a respective Node₁. Both Node₀ and Node₁ are shown in FIGS. 8, 10A, 10B, and 10C. Further, the secondary struts 428 may be additionally defined as a first secondary strut 428A and a second secondary strut 428B.

A first line of stitches 934, denoted by a solid line in FIGS. 10A and 10C, attaches the end segment 421 of the outer skirt 116 to each Y-shaped strut 422 as well as the second crowns 115. For ease of understanding, each Y-shaped strut 422 is numbered between 1 and 12 in FIGS. 10A, 10B, and 10C, the use of 12 Y-shaped struts 422 is merely exemplary and an alternative number of Y-shaped struts 422 are envisioned.

The first line of stitches 934 forms a repeating pattern that extends from the first Y-shaped strut 422 to the twelfth Y-shaped strut 422. In particular, the stitching pattern may begin at the first Y-shaped strut 422 and extend to the second Y-shaped strut 422 before repeating itself. Therefore, the pattern of the first line of stitches 934 may be described by discussing the pattern between the first Y-shaped strut 422 and the second Y-shaped strut 422. In order to provide clarity, the secondary struts 428 of the first Y-shaped strut 422 and the second Y-shaped strut 422 are additionally labeled as 428A, 428B, 428C, and 428D from left to right in FIGS. 10A, 10B, and 10C.

To describe the pattern between the first Y-shaped strut 422 and the second Y-shaped strut 422, a first segment of stitches 934A of the first line of stitches 934 axially extends along the secondary strut 428A towards the outflow end 412 (substantially vertically in FIG. 10A) from Node₁ to Node₀ of the first Y-shaped strut 422 as to couple the end segment 421 of the outer skirt 116 to the secondary strut 428A. In accordance with aspects hereof, the first segment of stitches 934A may begin either at Node₁ or at the secondary strut 428A substantially towards Node₁, and/or the first segment of stitches 934A may end at Node₀ or on the secondary strut 428A substantially towards Node₀. In some embodiments, the first segment of stitches 934A additionally may be described as an axial segment, or a vertical segment as it extends substantially parallel with the longitudinal axis L_{A} of the heart valve prosthesis 100.

A second segment of stitches 934B of the first line of stitches 934 extends circumferentially from Node₀ of the first Y-shaped strut 422 to Node₀ of the second Y-shaped strut 422 as to couple the end segment 421 of the outer skirt 116 to the first and second Y-shaped struts 422 at their respective Node₀ as well as to a respective second crown 115 that is disposed therebetween. In an aspect hereof, as shown in FIG. 10A, the second segment of stitches 934B may couple the end segment 421 to the second crown 115 that is positioned between the first Y-shaped strut 422 and the second Y-shaped strut 422. Further, the second segment of stitches 934B may couple the end segment 421 of the outer skirt 116 either at Node₀ of the second Y-shaped strut 422 or at the secondary strut 428D substantially towards Node₀. In some embodiments, the second segment of stitches 934B additionally may be described as a circumferential segment, or a horizontal segment as it extends around a portion of a circumference of the heart valve prosthesis 100.

A third segment of stitches 934C of the first line of stitches 934 axially extends along the secondary strut 428D towards the inflow end 410 (substantially vertically in FIG. 10A) from Node₀ of the second Y-shaped strut 422 to Node₁ at the opposite end of the secondary strut 428D. In accordance with aspects hereof, the third segment of stitches 934C may couple the end segment 421 of the outer skirt 116 either at Node₁ of the secondary strut 428D or at the secondary strut 428D substantially towards Node₁. In some embodiments, the third segment of stitches 934C additionally may be described as an axial segment, or a vertical segment as it also extends substantially parallel with the longitudinal axis LA of the heart valve prosthesis 100.

A fourth segment of stitches 934D of the first line of stitches 934 extends circumferentially from Node₁ at the end of the secondary strut 428D of the second Y-shaped strut 422 to an adjacent Node₁ of the third Y-shaped strut 422 as to couple the end segment 421 of the outer skirt 116 to the second and third Y-shaped struts 422 to at their respective Node₁. In some embodiments, the fourth segment of stitches 934D additionally may be described as a circumferential segment, or a horizontal segment as it also extends around a portion of a circumference of the heart valve prosthesis 100.

The first, second, third, and fourth segments of stitches 934A-934D are then repeated such that the first line of stitches 934 couples the end segment 421 of the outer skirt 116 to each of the Y-shaped struts 422 in a repeating pattern.

A second line of stitches 936, as denoted by a dashed line in FIGS. 10B and 10C, further attaches the end segment 421 of the outer skirt 116 to each Y-shaped strut 422 as well as the second crowns 115. As noted above, for ease of understanding each Y-shaped strut 422 is numbered between 1 and 12 in FIGS. 10A, 10B, and 10C.

The second line of stitches 936 forms a repeating pattern that extends from the first Y-shaped strut 422 to the twelfth Y-shaped strut 422. Similarly, to the first line of stitches 934, the stitching pattern begins at the first Y-shaped strut 422 and extends to the second Y-shaped strut 422 before repeating itself. Therefore, the pattern of the second line of stitches 936 may be described by discussing the pattern positioned between the first Y-shaped strut 422 and the second Y-shaped strut 422.

A first segment of stitches 936A of the second line of stitches 936 axially extends towards the inflow end 410 along the secondary strut 428B (substantially vertically in FIG. 10B) from Node₀ to Node₁ of the first Y-shaped strut 422 as to couple the end segment 421 of the outer skirt 116 to the secondary strut 428B. The first segment of stitches 936A of the second line of stitches 936 may be positioned at Node₀ of the Y-shaped strut 422 or may be positioned on the secondary strut 428B substantially towards Node₀. Further, the first segment of stitches 936A may be positioned either at Node₁ of the secondary strut 428B or on the secondary strut 428B substantially towards Node₁. In some embodiments, the first segment of stitches 936A additionally may be described as an axial segment, or a vertical segment as it also extends substantially parallel with the longitudinal axis L_{A} of the heart valve prosthesis 100.

A second segment of stitches 936B of the second line of stitches 936 extends circumferentially from Node₁ of the secondary strut 428B of first Y-shaped strut 422 to an adjacent Node₁ of the secondary strut 428C of the second Y-shaped strut 422 as to couple the end segment 421 of the outer skirt 116 to the first and second Y-shaped struts 422 at their respective Node₁. Further, the second segment of stitches 936B of the second line of stitches 936 may be positioned either at Node₁ of the secondary strut 428C or on the secondary strut 428C substantially towards Node₁. In some embodiments, the second segment of stitches 936B additionally may be described as a circumferential segment, or a horizontal segment as it also extends around a portion of a circumference of the heart valve prosthesis 100.

A third segment of stitches 936C of the second line of stitches 936 axially extends along the secondary strut 428C towards the outflow portion 412 (substantially vertically in FIG. 10B) from Node₁ of the second Y-shaped strut 422 to Node₀ at the opposite end of the secondary strut 428C. The third segment of stitches 936C of the second line of stitches 936 may couple the end segment 421 of the outer skirt 116 to either the Node₀ of the second Y-shaped strut 422 or on the secondary strut 428C substantially towards Node₀. In some embodiments, the third segment of stitches 936C additional may be described as an axial segment, or a vertical segment as it also extends substantially parallel with the longitudinal axis LA of the heart valve prosthesis 100.

A fourth segment of stitches 936D of the second line of stitches 936 extends circumferentially from Node₀ at the end of the secondary strut 428C of the second Y-shaped strut 422 to an adjacent Node₀ of the third Y-shaped strut 422 as to couple the end segment 421 of the outer skirt 116 to the second and third Y-shaped struts 422 to at their respective Node₀, as well as to a respective second crown 115 that is disposed therebetween. In some embodiments, the fourth segment of stitches 936D additionally may be described as a circumferential segment, or a horizontal segment as it also extends around a portion of a circumference of the heart valve prosthesis 100.

The first, second, third, and fourth segments of stitches 936A-936D are then repeated such that the second line of stitches 936 couples the end segment 421 of the outer skirt 116 to each of the Y-shaped struts 422 in a repeating pattern.

FIG. 10C shows the first line of stitches 934 and the second line of stitches 936 in combination coupling the end segment 421 of the outer skirt 116 to each of the Y-shaped struts 422. As shown in FIG. 10C, the pattern of the first line of stitches 934 and the pattern of second line of stitches 936 overlay each other so as to alternate first, second, third and fourth segments, allowing for the end segment 421 of the outer skirt 116 to be coupled to both Node₁ and the Node₀ of each Y-shaped strut 422.

In some embodiments, the first line of stitches 934 and the second line of stitches 936 may be made by one continuous suture 730. The first line of stitches 934 are created around the circumference of the outflow end 412 of the outer portion 108 and, once completed, the second set of stitches 936 is then created on a return path using the same, continuous, suture 730. Through the use of a single suture 730, the number of knots required is reduced, and, subsequently, decreases the deployment forces of the heart valve prosthesis 100 and decreases the potential of damage to the suture 730.

The sutures 730 may be a natural or biological material such as pericardium or another membranous tissue such as intestinal submucosa. Alternatively, the sutures may be a monofilament suture or of a low-porosity woven fabric, such as polyester, Dacron fabric, or PTFE. Further, the diameter of the suture may be configured to either increase the strength of the stitching or to reduce the packing volume of the heart valve prosthesis 100. For example, in some embodiments, using a suture having a diameter between 50 μm and 69 μm will reduce packing size of the heart valve prosthesis 100 while maintaining the strength to sufficiently couple the outer skirt 116 to the outer portion 108. In some embodiments, DYNEEMA 7-0 or 5-0 sutures may be used.

In order to create the heart valve prosthesis 100, the inner skirt 118 is first coupled to the inner portion 104. The outer skirt 116 is then coupled to the outer portion 108 using a plurality of sutures or adhesive. In particular, the outer skirt 116 is coupled to each Y-shaped strut 422 by first creating the first line of stitches 934 before then creating the second line of stitches 936. The combination of the first line of stitches 934 and the second line of stitches 936 secures the outer skirt 116 to each Node₁ and Node₀ of each Y-shaped strut 422 in such a manner as to reduce a tent height H₂ of the end segment 421 of the outer skirt 116 to reduce obstruction within the blood flow passageways 558 and thereby maximize blood flow through each of the blood flow passageways 558. After the outer skirt 116 is secured to the outer portion 108, the inner portion 104 and the outer portion 108 are then coupled to one another using rivets, soldering, or other methods known in the art within the apertures 348, 415 of the crowns 114, 346.

Although in the preceding description of specific embodiments of the present invention the stitching pattern is described as being used at an outflow end of the prosthesis, it is not so limited in applicability and is also beneficial for use at an inflow end of a prosthesis in various applications. More particularly, a stitching pattern in accordance herewith may be utilized to secure a skirt or liner at an inflow end of a frame for a prothesis when a blood flow passageway is needed to be maintained substantially unobstructed therethrough. Accordingly, use of “first end” and “second end” in the claims may be interpreted to refer to one of an “inflow end” and an “outflow end,” or vice versa, without departing from the scope of the present disclosure.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

What is claimed is:

1. A heart valve prosthesis comprising:
a frame including
an inner portion configured to support a prosthetic valve component and
an outer portion coupled to the inner portion, the outer portion being sized to surround the inner portion and configured to anchor the prosthesis, the outer portion including a plurality of first crowns positioned around a first end of the outer portion and a plurality of second crowns positioned inward of the plurality of first crowns; and
a skirt disposed within and coupled to the outer portion, wherein an end segment of the skirt, disposed proximate to the first end of the outer portion, does not substantially extend past the plurality of second crowns of the outer portion so as to leave a substantially unobstructed blood flow passageway through each of the pair of adjoining crowns.

2. The prosthesis of claim 1, wherein the outer portion includes a plurality of Y-shaped struts, each coupled to one of the plurality of first crowns and positioned proximate the first end.

3. The prosthesis of claim 2, wherein each of the Y-shaped struts includes
a node,
a primary strut that extends between a respective first crown of the plurality of first crowns and the node, and
a pair of secondary struts branching, at the node, from the primary strut toward the second end of the outer portion.

4. The prosthesis of claim 3, wherein the skirt is coupled to each Y-shaped strut and each second crown by a suture.

5. The prosthesis of claim 4, wherein the skirt is coupled to the primary strut and each of the pair of secondary struts of each Y-Shaped strut.

6. The prosthesis of claim 3, wherein a first line of stitches couples the skirt to a first portion of each Y-shaped strut and a second line of stitches couples the skirt to a second portion of each Y-shaped strut.

7. The prosthesis of claim 6, wherein each of the first line of stitches and the second line of stitches forms a repeating pattern of axial segments and circumferential segments.

8. The prosthesis of claim 7, wherein the first line of stitches and the second line of stitches are completed with a single continuous suture.

9. A method for manufacturing a heart valve prosthesis comprising:
attaching an inner skirt to an inner portion;
attaching an outer skirt to an outer portion,
coupling the inner portion to the outer portion, wherein a plurality of pairs of adjoining crowns are defined at the points where the inner portion and the outer portion are coupled; and
wherein an end segment of the outer skirt, disposed proximate to a first end of the outer portion, does not substantially extend past a plurality of second crowns of the outer portion as to leave a substantially unobstructed blood flow passageway through each pair of adjoining crowns.

10. The method of claim 9, wherein the outer portion has a stent-like structure with a second end opposite the first end, the outer portion including a plurality of first crowns, a plurality of second crowns, and plurality of Y-shaped strut positioned at the first end.

11. The method of claim 10, wherein each of the Y-shaped struts includes
a node,
a primary strut is positioned between a respective first crown of the plurality of first crowns and the node, and
a pair of secondary struts branching, at the node, from the primary strut toward the second end of the outer portion.

12. The method of claim 11, wherein the outer skirt is coupled to each Y-shaped struts and each second crown by a suture.

13. The method of claim 12, wherein the outer skirt is coupled to the primary strut and each of the pair of secondary struts of each Y-Shaped strut.

14. The method of claim 12, wherein a first line of stitches couples the skirt to a first portion of each Y-shaped strut and a second line of stitches couples the skirt to a second portion of each Y-shaped strut.

15. The method of claim 14, wherein each of the first line of stitches and the second line of stitches forms a repeating pattern of axial segments and circumferential segments.

16. The method of claim 14, wherein the first line of stitches and the second line of stitches are completed with a single continuous suture.
